(19) United States
(12) Patent Application Publication
(10) Pub. No.: US 2024/0115833 A1
(43) Pub. Date: Apr. 11, 2024

(54) CATHETER WITH REDUCED CONTACT SURFACE

(71) Applicant: DePuy Synthes Products, Inc., Rayham, MA (US)

(72) Inventor: Pedro D. PEDROSO, Raynham, MA (US)

(73) Assignee: DePuy Synthes Products, Inc., Raynham, MA (US)

(21) Appl. No.: 17/958,651

(22) Filed: Oct. 3, 2022

(51) Int. Cl. A61M 25/00

(52) U.S. Cl. CPC A61M 25/0043; A61M 2025/0042; A61M 2025/006; A61M 2210/12

(57) ABSTRACT

The objectives for the designs presented herein can be for a variably flexible catheter for vascular applications. The catheter can include a catheter shaft extending along a longitudinal axis. The catheter can include an exterior surface that includes an exterior surface area. The catheter can include a plurality of ridges each including an apex, an amplitude, a wavelength, and a trough with respect to the longitudinal axis. The apexes of the plurality of ridges can define a contact exterior surface area less than the exterior surface area. When the exterior surface of the catheter is applied to vascular tissue, contact between the vascular tissue made primarily by the contact exterior surface area.

### FIELD OF THE INVENTION

The present disclosure generally relates to devices and methods for accessing blood vessels during intravascular medical treatments. More specifically, the present disclosure relates to a catheter having improved flexibility and reduced contact surfaces for improved tracking in tortuous anatomy.

### BACKGROUND

Catheters serve a broad range of functions in intravascular medical treatments. Catheters are typically a thin tube manufactured from medical grade materials that can be inserted into a body and can be used to deliver drugs or other devices, perform surgical procedures, remove blockages from vessels, and a variety of other purposes. By modifying the material or adjusting the way a catheter is manufactured, it is possible to tailor different sections of the catheter for particular applications.

There are a number of access challenges that can make it difficult to access a target site. In cases where access involves navigating the aortic arch (such as with coronary or cerebral blockages) the configuration of the arch in some patients makes it difficult to position a guide catheter. Beyond the arch, accessing the neurovascular bed in particular is challenging with conventional technology, as the target vessels are small in diameter, remote relative to the site of insertion, and are highly tortuous. It is not unusual that a catheter will have to navigate winding pathways with multiple loops, where vessel segment can have several extreme bends in quick succession over only a few centimeters of travel, which can lead to kinking. The ever-narrower reaches of the arterial system can have delicate vessels that can easily be damaged by inflexible or high-profile devices.

Catheters for these procedures can be difficult to design in that they must be fairly stiff at the proximal end to maintain pushability and responsive yet comfortable manipulation for the user, while having the flexibility in more distal portions to endure high flexure strains and progress through loops and increasingly smaller vessel sizes without causing trauma. For these reasons, size, kink-resistance, trackability, and flexibility are the key design parameters usually associated with catheters used in these procedures and managing the transition from softer to stiffer materials and regions is critical to successful patient outcomes.

Several designs and methods have been proposed for getting a catheter to a target site. In one method, the catheter fits over and is slid along a guidewire which is used to gain access to a target site. A thin guidewire, however, almost always has more reach and distal flexibility than the catheter tube. Newer designs have been proposed which utilize various methods to alter the stiffness between the proximal and distal portions of the catheter, such as sets of polymer tubing, often with braids or windings involving wires or bands of other materials for reinforcement.

Additionally, in thrombectomy procedures, aspiration catheters need to be very flexible to access a remote occlusion, but also benefit from good compressive stiffness (for pushability, and stability and integrity when clot retrieval devices are withdrawn into them) and good tensile stiffness (to avoid stretch and deformation when placed in tension, such as when being retrieved into an outer sheath while holding a large clot). It has been tricky for designers of traditional catheters to combine these characteristics without large trade-offs. Thus, catheter design has often leaned towards sacrificing proximal column strength to gain in flexibility and trackability.

The present designs are aimed at providing an improved catheter construction to address the above-stated deficiencies.

### SUMMARY

The innovations of this disclosure involve a catheter which has features for reducing the contact surface of the catheter shaft against the vasculature, as well as reducing the contact surface of the interior lumen of the catheter for improved tracking and positioning of devices being delivered through the interior lumen of the catheter shaft. The catheter can include an exterior surface forming an exterior surface area. The catheter can include a plurality of ridges each having an apex, an amplitude, a wavelength, and a trough. The apexes of the plurality of ridges can define a contact exterior surface area. When the exterior surface of the catheter is shaft is applied to vascular tissue, contact between the vascular tissue is made primarily by the contact exterior surface area.

The disclosed designs can have the ability to vary the stiffness along the length of the catheter shaft by varying the wavelengths of the plurality of ridges along the length of the catheter without the use of braided members with varying braid counts or coil pitches and without changing the durometer hardness of the surrounding polymer material.

In some examples, the apexes of the plurality of ridges can define an external circumference of the catheter shaft and the troughs can define an internal circumference of the catheter shaft.

In some examples, the catheter shaft can include a first section having a first plurality of ridges that have a first wavelength and a second section having a second plurality of ridges having a second wavelength. The first plurality of ridges and the second plurality of ridges can have equivalent amplitudes and cross sectional thicknesses.

In some examples, the first wavelength of the first plurality of ridges and the second wavelength of the second plurality of ridges are effective to control a stiffness of the catheter shaft.

In some examples, the first wavelength is greater than the second wavelength and the first section has a lower stiffness than the second section.

In some examples, the catheter shaft can include an interior surface that has a total interior surface area. The troughs of the plurality of ridges can define a contact interior surface area less than the total interior surface area.

In some examples, the amplitude is measured from the apex to the trough of a respective ridge of the plurality of ridges.

In another aspect, a catheter shaft is disclosed that can extend along a longitudinal axis and can include an exterior surface having an exterior surface area. The catheter shaft can include a plurality of ridges each having an apex, an amplitude, a wavelength, and a trough with respect to the longitudinal axis. The apexes of the plurality of ridges can define a contact exterior surface area less than the exterior surface area. The catheter shaft can include an interior surface that has a total interior surface area. The troughs of the plurality of ridges can define a contact interior surface area that is less than the total interior surface area.

In some examples, the apexes of the plurality of ridges can define an external circumference of the catheter shaft and the troughs can define an internal circumference of the catheter shaft.

In some examples, the catheter shaft can include a first section that has a first plurality of ridges having a first wavelength. The catheter shaft can include a second section that has a second plurality of ridges that have a second wavelength. The first plurality of ridges and the second plurality of ridges can have equivalent amplitudes and cross sectional thicknesses.

In some examples, the first wavelength of the first plurality of ridges and the second wavelength of the second plurality of ridges are effective to control a stiffness of the catheter shaft.

In some examples, the first wavelength is greater than the second wavelength.

In some examples, the first section has a lower stiffness than the second section.

In some examples the amplitude is measured from the apex to the trough of a respective ridge of the plurality of ridges.

In another aspect, a method of treating a medical condition with a catheter is disclosed. The method can include providing a catheter shaft having a plurality of ridges on an exterior surface of the catheter shaft. Each of the plurality of ridges can include an apex, an amplitude, a wavelength, and a trough with respect to the longitudinal axis of the catheter shaft. The method can include inserting the catheter shaft into vasculature of a patient. The method can include making contact with the vasculature with primarily the apexes of the plurality of ridges.

In some examples, the catheter shaft can include a first section and a second section. The first section can have a first plurality of ridges including a first wavelength and the second section can have a second plurality of ridges that have a second wavelength.

In some examples, a rigidity of the catheter shaft can be different between the first section and the second section due to a difference between the first wavelength and the second wavelength.

In some examples, the first plurality of ridges and the second plurality of ridges have equivalent amplitudes and cross sectional thicknesses.

In some examples, the first wavelength is greater than the second wavelength and the first section has a lower stiffness than the second section.

In some examples, the method can further include inserting a microcatheter into a lumen of the catheter shaft and making contact with the microcatheter with primarily the trough of the plurality of ridges.

Other aspects and features of the present disclosure will become apparent to those of ordinary skill in the art, upon reviewing the following detailed description in conjunction with the accompanying figures.

### BRIEF DESCRIPTION OF THE DRAWINGS

The above and further aspects of this invention are further discussed with reference to the following description in conjunction with the accompanying drawings. The drawings are not necessarily to scale, emphasis instead being placed upon illustrating the principles of the invention. The figures depict one or more implementations of the inventive devices, by way of example only, not by way of limitation.

FIG. 1A is a perspective illustration of a catheter which has a reduced contact surface formed by a first plurality of ridges, according to aspects of the present invention;

FIG. 1B is a cross sectional view of the catheter of FIG. 1A, according to aspects of the present invention;

FIG. 1C illustrates a sample ridge section of the catheter of FIGS. 1A-1B, according to aspects of the present invention;

FIG. 2A is a perspective illustration of a catheter which has a reduced contact surface formed by a second plurality of ridges, according to aspects of the present invention;

FIG. 2B is a cross sectional view of the catheter of FIG. 2A, according to aspects of the present invention;

FIG. 2C illustrates a sample ridge section of the catheter of FIGS. 2A-2B, according to aspects of the present invention;

FIG. 3 illustrates a catheter having two sections with varied rigidity by varying the wavelength of the plurality of ridges along the length of the catheter, according to aspects of the present invention; and

FIG. 4 is a flowchart of a method of treating a medical condition with a catheter, according to aspects of the present invention.

### DETAILED DESCRIPTION

The objectives for the designs presented herein can be for a variably flexible and kink-resistant elongated catheter shafts for vascular applications. The designs are flexible enough to access remote vessel occlusions but also benefit from good compressive and tensile stiffness. The designs also allow for a catheter with varied stiffness throughout lengths of the catheter without the use of braided members with varying braid counts or coil pitches and without changing the durometer hardness of the surrounding polymer material.

While the description is in many cases in the context of mechanical thrombectomy or other treatments in the neurovascular bed, the devices and methods described may be easily adapted for other procedures and in other body passageways where a catheter with a highly adaptable stiffness requirement is needed. For example, microcatheters typically having a much smaller diameter than other catheters can also be made using these concepts.

Accessing the various vessels within the vascular, whether they are coronary, pulmonary, or cerebral, involves well-known procedural steps and the use of a number of conventional, commercially available accessory products. These products can involve angiographic materials, rotating hemostasis valves, and guidewires as widely used in laboratory and medical procedures. Though they may not be mentioned specifically by name, when these or similar products are necessarily employed in conjunction with the system and methods of this invention in the description below, their function and exact constitution are not described in detail.

Specific examples of the present invention are now described in detail with reference to the Figures, where identical reference numbers indicate elements which are functionally similar or identical.

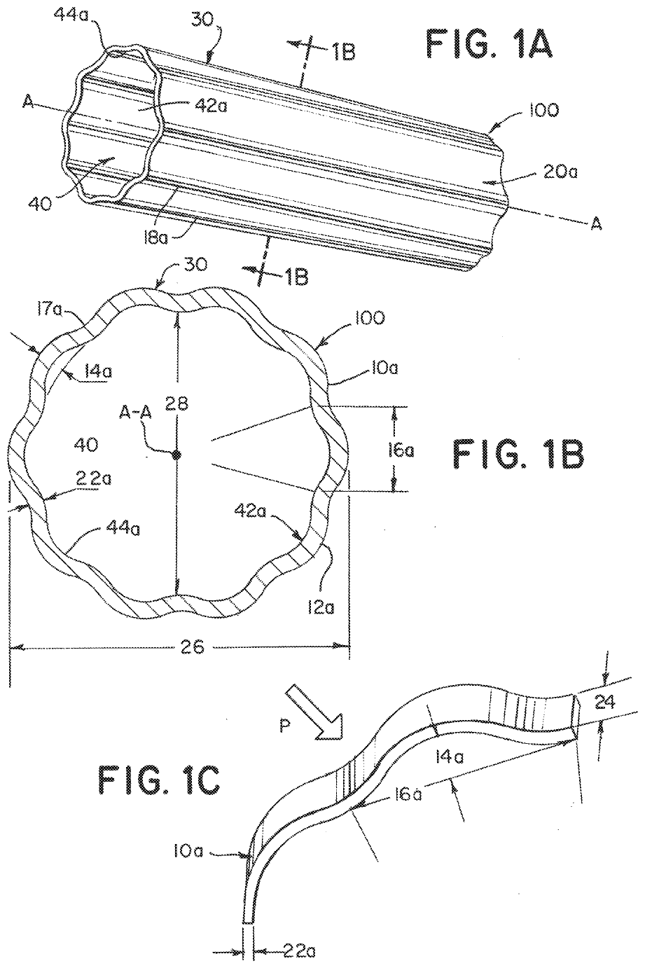

Turning to the figures, in FIG. 1A there is illustrated a catheter section 100 which has a reduced contact surface formed by a plurality of ridges 10a. Catheter section 100 can be used in intravascular procedures in the vessels of a patient. Catheter section 100 can include an exterior surface 30 with total exterior surface area 20a and a contact exterior surface area 18a that is less than the total exterior surface area 20a. Catheter section 100 can also include an interior surface 40 with a total interior surface area 42a and a contact interior surface area 44a that is less than the total interior surface area 42a. Catheter section 100 also includes a longitudinal axis A-A running axially through the length of catheter section 100. The wall of catheter section 100 can be formed of PTFE or other low friction material to facilitate the passage of ancillary devices through the catheter lumen, as well as facilitate the pushability of catheter section 100 through vasculature. The wall of catheter section 100 can have a wall thickness 22a (FIG. 1C), which can be varied according to modify desired properties, such as stiffness of catheter section 100.

FIG. 1B illustrates a cross-sectional view of the catheter of FIG. 1A. The plurality of ridges 10a include apexes 12a, amplitudes 14a, troughs 17a, and wavelength 16a. The troughs 17a define an internal circumference 28, and the apexes 12a define an external circumference 26. The wavelength 16a can be measured between adjacent troughs 17a and/or between adjacent apexes 12a. When tracking catheter section 100 through vasculature, contact between the vasculature and the catheter section 100 is made by the contact exterior surface area 18a which is less than the total exterior surface area 20a. Contact exterior surface area 18a is defined by the apexes 12a. That is, primarily the apexes 12a make contact with the surrounding vasculature while catheter section 100 is in use. Similarly, when ancillary devices are pushed through catheter section 100, the contact between the ancillary device (not shown) and the catheter section 100 is made by contact interior surface area 44a which is less than the total interior surface area 42a. Contact interior surface area 44a is defined by the troughs 17a. That is, primarily the troughs 17a make contact with the ancillary device while ancillary device is pushed through catheter section 100.

FIG. 1C illustrates a sample ridge section of the catheter of FIGS. 1A-1B. As shown, the sample ridge 10a has an amplitude 14a, wavelength 16a, width 24 and thickness 22a. The rigidity of a length of catheter section 100 can be controlled by varying one or more features of the ridge 10a. Modeling the sample ridge 10a shown in FIG. 1C as a deflecting supported beam in a three point configuration, the relative flexural force “P” needed to deform this section can be varied according to the following relationship, provided in Equation (1):

$$P = \frac{4 {d}_{f} E S {w}^{3}}{{L}^{3}} \qquad ( 1 )$$

In Equation (1), P stands for the flexural force needed to deform a given length, d_{f} gives the amplitude (e.g., amplitude 14a), E stands for relative Young's modulus, w stands for thickness (e.g., thickness 22a), S stands for width (e.g., width 24), and L stands for wavelength (e.g., wavelength 16a). In some examples, by varying the wavelength L (e.g. 16a) and leaving the other factors constant, portions of the catheter section 100 length can have varying stiffness levels without changing the stiffness of material used, which improves ease of manufacturing. In other examples, other geometries such as amplitude d_{f} (e.g. 14a) can be varied throughout the length of the catheter in order to change the stiffness of the catheter section 100.

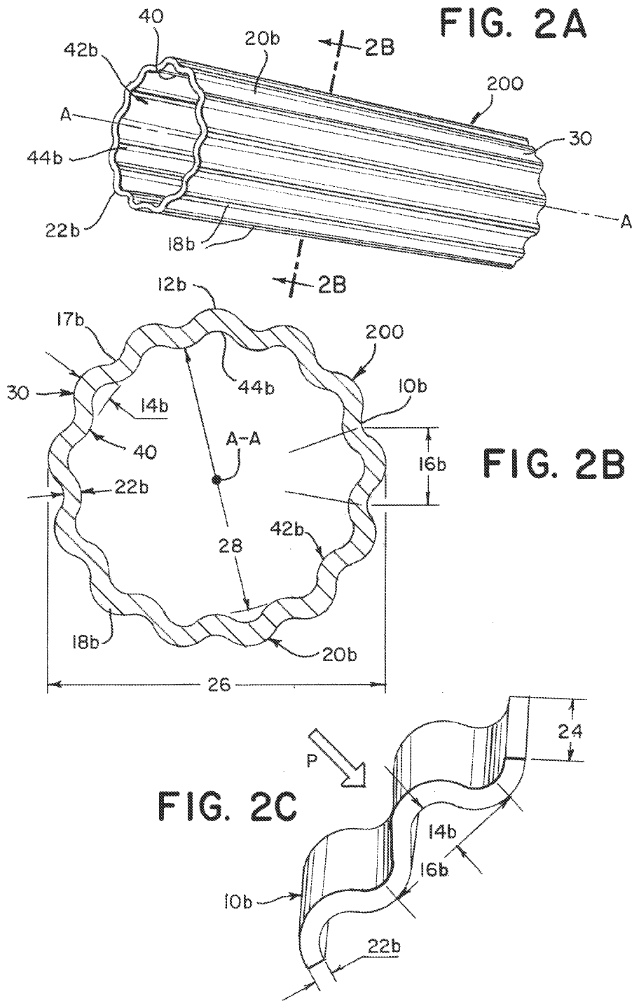

In FIG. 2A there is illustrated a catheter section 200 which has a reduced contact surface formed by a plurality of ridges 10b. Catheter section 200 can be used in intravascular procedures in the vessels of a patient. Catheter section 200 can include an exterior surface 30 with total exterior surface area 20b and a contact exterior surface area 18b that is less than the total exterior surface area 20b. Catheter section 200 can also include an interior surface 40 with a total interior surface area 42b and a contact interior surface area 44b that is less than the total interior surface area 42b. Catheter section 200 also includes a longitudinal axis A-A running axially through the length of catheter section 200. The wall of catheter section 200 can be formed of PTFE or other low friction material to facilitate the passage of ancillary devices through the catheter lumen, as well as facilitate the pushability of catheter section 200 through vasculature. The wall of catheter section 200 can have a wall thickness 22b (FIG. 2C), which can be varied according to modify desired properties, such as stiffness of catheter section 200.

FIG. 2B illustrates a cross-sectional view of the catheter of FIG. 2A. The plurality of ridges 10b include apexes 12b, amplitudes 14b, troughs 17b, and wavelength 16b. The troughs 17b define an internal circumference 28, and the apexes 12b define an external circumference 26. The wavelength 16b can be measured between adjacent troughs 17b and/or between adjacent apexes 12b. When tracking catheter section 200 through vasculature, contact between the vasculature and the catheter section 200 is made by the contact exterior surface area 18b which is less than the total exterior surface area 20b. Contact exterior surface area 18b is defined by the apexes 12b. That is, primarily the apexes 12b make contact with the surrounding vasculature while catheter section 200 is in use. Similarly, when ancillary devices are pushed through catheter section 200, the contact between the ancillary device (not shown) and the catheter section 200 is made by contact interior surface area 44b which is less than the total interior surface area 42b. Contact interior surface area 44b is defined by the troughs 17b. That is, primarily the troughs 17b make contact with the ancillary device while ancillary device is pushed through catheter section 200.

FIG. 2C illustrates a sample ridge section of the catheter of FIGS. 2A-2B. As shown, the sample ridge 10b has an amplitude 14b, wavelength 16a, width 24 and thickness 22a. The rigidity of a length of catheter section 200 can be controlled by varying one or more features of the ridge 10. Modeling the sample ridge 10b shown in FIG. 2C as a deflecting supported beam in a three point configuration, the relative flexural force “P” needed to deform this section can be varied according to Equation (1). In Equation (1), P stands for the flexural force needed to deform a given length, d_{f} gives the amplitude (e.g., amplitude 14b), E stands for relative Young's modulus, w stands for thickness (e.g., thickness 22b), s stands for width (e.g., width 24), and L stands for wavelength (e.g., wavelength 16b). In some examples, by varying the wavelength L (e.g. 16b) and leaving the other factors constant, portions of the catheter section 200 length can have varying stiffness levels without changing the stiffness of material used, which improves ease of manufacturing. In other examples, other geometries such as amplitude d_{f} (e.g. 14b) can be varied throughout the length of the catheter in order to change the stiffness of the catheter section 200.

In contrast to the example illustrated in FIGS. 1A-1C, the example catheter section 100 of FIGS. 2A-2C has a smaller wavelength L, which as per Equation (1), causes the catheter section 200 to have a higher stiffness (e.g., by increasing the denominator value of L 3, the value of P in Equation (1) increases).

Generally, fewer ridges can result in a more flexible catheter section and more ridges can result in a stiffer catheter section. Therefore, more ridges can be used in a proximal section of the catheter to facilitate pushing of the catheter, and fewer ridges can be used in a distal section of the catheter to provide greater flexibility. A catheter section preferably has three to twenty ridges such that a catheter includes sections having any number of ridges between and including three and twenty ridges. More preferably, a catheter section has six to sixteen ridges. More preferably, a catheter section has ten to thirteen ridges as illustrated.

FIG. 3 illustrates a catheter 300 having two sections with varied rigidity by varying the wavelength of the plurality of ridges (e.g., ridges 10a and ridges 10b) along the length of the catheter 300. Catheter 300 can include two sections, first section 110 and second section 210. The first section 110 can include a plurality of ridges 10a similar to the catheter section 100 as described with respect to FIGS. 1A-1C and the second section 210 can include a plurality of ridges 10b similar to the catheter section 200 as described with respect to FIGS. 2A-2C. In this way, catheter 300 can have varying stiffness between first section 110 and second section 210. The first section 110 can have a cross-section similar to as illustrated in FIG. 1B, and the section 210 can have a cross-section similar to as illustrated in FIG. 2B.

The first section 110 can be less stiff than the second section 210 because the plurality of ridges 10b in the second section 210 can have a smaller wavelength 16b as compared to wavelength 16a in the first section 110. Although catheter 300 is shown having a first section 110 and a second section 210, a catheter can be constructed using these principles with any number of catheter sections with varying degrees of stiffness by modifying not only wavelength 16a, 16b of the plurality of ridges, but also any of the other variables given in Equation (1) (e.g., amplitude 14a, 14b, thickness 22a, 22b, width 24a, 24b, etc.). Catheter 300 keeps the desirable properties of catheter sections 100 and 200, such as decreased friction between vessels externally and ancillary devices internally, and improved pushability through the vasculature, while also providing the benefit of varying levels of stiffness throughout the catheter by varying properties of the plurality of ridges without the added complexity of introducing different materials to the design.

The transition between the first section 110 and the second section 210 can be abrupt; however, the transition is more preferably gradual, where the wave pattern is shifted and wavelengths are added in a tapered fashion over a length. The transition can be gradual in that one wavelength is added over a first length, and a second wavelength is added over a second length distal to the first length. Alternatively, multiple wavelengths can be added over a single length in a tapered fashion.

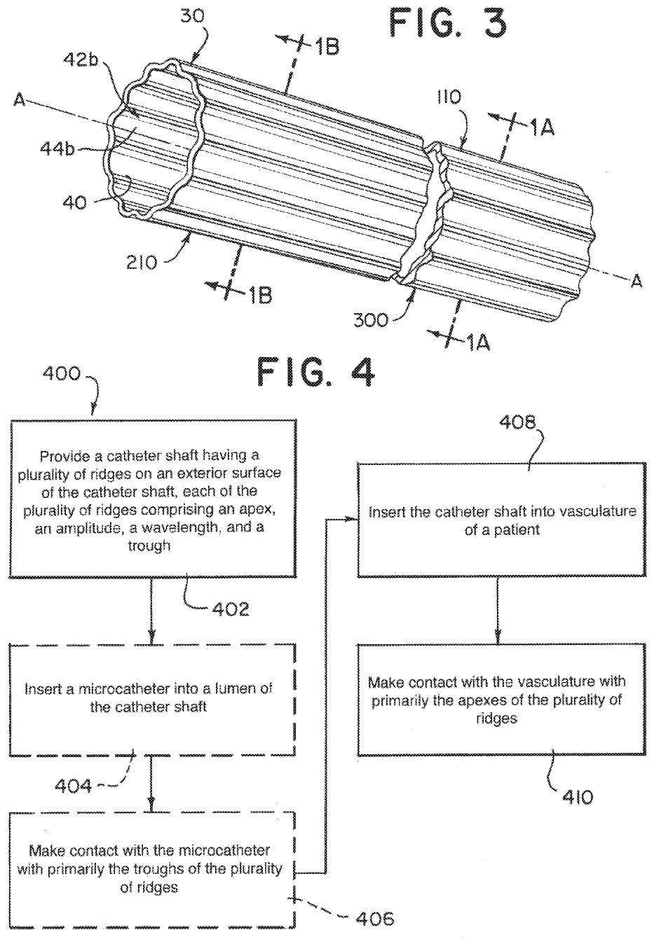

FIG. 4 is a flowchart of a method 400 of treating a medical condition with a catheter. In block 402, the method can include providing a catheter shaft (e.g., catheter sections 100, 200, and/or catheter 300) having a plurality of ridges 10 on an exterior surface of the catheter shaft. Each of the plurality of ridges can include an apex 12a, 12b, an amplitude 14a, 14b, a wavelength 16a, 16b, and a trough 17a, 17b with respect to a longitudinal axis A-A of the catheter shaft 100, 200, 300.

In optional block 404, the method can include inserting a microcatheter 600 into a lumen of the catheter shaft 100, 200, 300. In optional block 406, the method can include making contact with the microcatheter 600 with primarily the troughs 17a, 17b of the plurality of ridges.

In block 408, the method can include inserting the catheter shaft 100, 200, 300 into vasculature of a patient. In block 410, the method can include making contact with the vasculature with primarily the apexes 12a, 12b of the plurality of ridges 10a, 10b.

In some examples, the catheter shaft 300 can include a first section 110 and a second section 210. The first section 110 can have a first plurality of ridges 10a with first wavelength 16a, and the second section can have a second plurality of ridges 10b that have a second wavelength 16b.

In some examples, the first plurality of ridges 10a and the second plurality of ridges 10b can have equivalent amplitudes 14a, 14b and cross sectional thicknesses 22a, 22b.

In some examples, the first wavelength 16a can be greater than the second wavelength 16b, and the first section 110 can have a lower stiffness than the second section 210.

The invention is not necessarily limited to the examples described, which can be varied in construction and detail. The terms “distal” and “proximal” are used throughout the preceding description and are meant to refer to a positions and directions relative to a treating physician. As such, “distal” or distally” refer to a position distant to or a direction away from the physician. Similarly, “proximal” or “proximally” refer to a position near to or a direction towards the physician. Furthermore, the singular forms “a,” “an,” and “the” include plural referents unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±20% of the recited value, e.g. “about 90%” may refer to the range of values from 71% to 99%.

In describing example embodiments, terminology has been resorted to for the sake of clarity. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology. For clarity and conciseness, not all possible combinations have been listed, and such variants are often apparent to those of skill in the art and are intended to be within the scope of the claims which follow.

What is claimed is:

1. A catheter shaft extending along a longitudinal axis comprising:
an exterior surface comprising an exterior surface area;
a plurality of ridges each comprising, an apex, an amplitude, a wavelength, and a trough with respect to the longitudinal axis; and
the apexes of the plurality of ridges defining a contact exterior surface area less than the exterior surface area,
wherein, when the exterior surface of the catheter shaft is applied to vascular tissue, contact between the vascular tissue is made primarily by the contact exterior surface area.

2. The catheter shaft of claim 1, the apexes of the plurality of ridges defining an external circumference of the catheter shaft and the troughs defining an internal circumference of the catheter shaft.

3. The catheter shaft of claim 1, further comprising:
a first section comprising a first plurality of ridges comprising a first wavelength; and
a second section comprising a second plurality of ridges comprising a second wavelength,
wherein the first plurality of ridges and the second plurality of ridges have equivalent amplitudes and cross sectional thicknesses.

4. The catheter shaft of claim 3, wherein the first wavelength of the first plurality of ridges and the second wavelength of the second plurality of ridges are effective to control a stiffness of the catheter shaft.

5. The catheter shaft of claim 4, wherein the first wavelength is greater than the second wavelength and the first section has a lower stiffness than the second section.

6. The catheter shaft of claim 1, further comprising:
an interior surface comprising a total interior surface area,
wherein the troughs of the plurality of ridges define a contact interior surface area less than the total interior surface area.

7. The catheter shaft of claim 1, wherein the amplitude is measured from the apex to the trough of a respective ridge of the plurality of ridges.

8. A catheter shaft extending along a longitudinal axis comprising:
an exterior surface comprising an exterior surface area;
a plurality of ridges each comprising an apex, an amplitude, a wavelength, and a trough with respect to the longitudinal axis, the apexes of the plurality of ridges defining a contact exterior surface area less than the exterior surface area; and
an interior surface comprising a total interior surface area, the troughs of the plurality of ridges defining a contact interior surface area less than the total interior surface area.

9. The catheter shaft of claim 8, the apexes of the plurality of ridges defining an external circumference of the catheter shaft and the troughs defining an internal circumference of the catheter shaft.

10. The catheter shaft of claim 8, further comprising:
a first section comprising a first plurality of ridges comprising a first wavelength; and
a second section comprising a second plurality of ridges comprising a second wavelength,
wherein the first plurality of ridges and the second plurality of ridges have equivalent amplitudes and cross sectional thicknesses.

11. The catheter shaft of claim 10, wherein the first wavelength of the first plurality of ridges and the second wavelength of the second plurality of ridges are effective to control a stiffness of the catheter shaft.

12. The catheter shaft of claim 11, wherein the first wavelength is greater than the second wavelength.

13. The catheter shaft of claim 12, wherein the first section has a lower stiffness than the second section.

14. The catheter shaft of claim 8, wherein the amplitude is measured from the apex to the trough of a respective ridge of the plurality of ridges.

15. A method of treating a medical condition with a catheter, comprising:
providing a catheter shaft having a plurality of ridges on an exterior surface of the catheter shaft, each of the plurality of ridges comprising an apex, an amplitude, a wavelength, and a trough with respect to a longitudinal axis of the catheter shaft;
inserting the catheter shaft into vasculature of a patient; and
making contact with the vasculature with primarily the apexes of the plurality of ridges.

16. The method of claim 15, wherein the catheter shaft comprises a first section and a second section, the first section having a first plurality of ridges comprising a first wavelength and the second section having a second plurality of ridges comprising a second wavelength.

17. The method of claim 16, wherein a rigidity of the catheter shaft is different between the first section and the second section due to a difference between the first wavelength and the second wavelength.

18. The method of claim 16, wherein the first plurality of ridges and the second plurality of ridges have equivalent amplitudes and cross sectional thicknesses.

19. The method of claim 16, wherein the first wavelength is greater than the second wavelength, and the first section has a lower stiffness than the second section.

20. The method of claim 15, further comprising:
inserting a microcatheter into a lumen of the catheter shaft; and
making contact with the microcatheter with primarily the troughs of the plurality of ridges.
